(19) United States
(12) Patent Application Publication
(10) Pub. No.: US 2008/0083413 A1
(43) Pub. Date: Apr. 10, 2008

(54) Detection of implanted injection port

(75) Inventor: Peter Forsell, Zug (CH)

Correspondence Address: NIXON & VANDERHYE, PC, 901 NORTH GLEBE ROAD, 11TH FLOOR, ARLINGTON, VA, 22203, US

(73) Assignee: Potencia Medical AG, Baar (CH)

(21) Appl. No.: 11/987,723

(22) Filed: Dec. 4, 2007

Related U.S. Application Data
(63) Continuation of application No. 10/530,036, filed on Aug. 17, 2005.
Application No. 10/530,036 is a continuation-in-part of application No. 10/260,546, filed on Oct. 1, 2002, now abandoned.

(51) Int. Cl. A61B 19/00

(52) U.S. Cl. 128/899

(57) ABSTRACT

An apparatus for detecting an injection port subcutaneously implanted in a patient includes a magnetic device, such as a permanent magnet or a solenoid, that emits a local magnetic field, and a magnetic detector, preferably including at least one Hall element, that detects the local magnetic field. The magnetic device is designed to be subcutaneously implanted in the patient at the implanted injection port, and the magnetic detector is movable externally along the patient's body to establish an injection position at the patient's skin in front of the implanted injection port where the local magnetic field emitted by the magnetic device is detected by the magnetic detector. As a result, an injection needle can be placed in the established injection position, in order to insert the injection needle through the patient's skin directly into the injection port substantially in the centre thereof.

The present invention relates to an apparatus and a method for detecting an injection Dort subcutaneously implanted in a patient.

It is important to locate the position of an injection port connected to a hydraulically operable surgical implant in a patient to be able to accurately inject a needle of a syringe through the membrane of the injection port (or simply for the purpose of locating the exact position of the injection port, or alternatively locating the membrane of the injection port), for supplying hydraulic fluid to or withdrawing hydraulic fluid from the implant. Such an injection port is typically arranged in connection (via a conduit) to a hydraulically adjustable implant, for example a food intake restriction device, implanted inside an obese patient.

Currently, a nurse or doctor locates an implanted injection port by simply feeling with the fingers on the patient's skin to find out where the injection port, is situated. However, the nurse or doctor cannot know exactly where the injection needle should penetrate the skin, in order to penetrate the centre of the membrane of the injection port.

The object of the present invention is to provide an inexpensive apparatus and methods for accurately detecting an injection port subcutaneously implanted in a patient to enable an injection needle to safely penetrate the patient's skin directly into the centre of the injection port.

This object is obtained by an apparatus of the kind stated initially, comprising a magnetic device adapted to emit a local magnetic field, and a magnetic detector adapted to detect the local magnetic field emitted by the magnetic device. The magnetic device is designed to be subcutaneously implanted in the patient at the implanted injection port, and the magnetic detector is movable externally along the patient's body to establish an injection position at the patient's skin in front of the implanted injection port where the local magnetic field emitted by the magnetic device is detected by the magnetic detector. Alternatively, the magnetic detector is designed to be subcutaneously implanted and the magnetic device is movable along the patient's body to establish the injection position at the patient's skin in front of the implanted injection port where the local magnetic field emitted by the magnetic device is detected by the magnetic detector.

Thus, the accurate injection position on the patient's skin in front of the injection port, which is hidden behind the skin, can be established using the apparatus of the present invention. With an injection needle placed in this injection position, it is an easy task to properly and safely insert the injection needle through the patient's skin directly into the injection port substantially in the centre thereof. The present invention is particularly advantageous to practise in obese people where an implanted injection port can be difficult to manually locate.

Generally, the magnetic detector Includes a semiconductor circuit, preferably in the form of at least one Hall-element. By using one or more Hall-elements, a special type of semiconductor known in the art, it is easy to locate the centre of the magnetic field emitted by the magnetic device. The magnetic detector suitably comprises several Hall-elements grouped around a central point in a triangular or square configuration. For example, three Hall-elements may be arranged at the corners of an equilateral triangle. An important advantage is that the Hall-elements are able to detect even a weak magnetic field emitted from the magnetic device.

In accordance with a main first embodiment of the invention, the magnetic device is designed to be subcutaneously implanted in the patient at the implanted injection port to emit the local magnetic field through a portion of the patient's skin adjacent to the injection port, and the magnetic detector is movable eternally along the patient's body to establish the injection position where the local magnetic field is detected by the magnetic detector. In this embodiment, the magnetic device may include a ring-magnet to be positioned around the membrane o the injection port, so that an injection needle can be inserted through the ring-magnet and the membrane of the injection port. The magnetic detector may be adapted to emit a sound when detecting the local magnetic field. Alternatively, the movable magnetic detector may be provided with at least one diode adapted to emit light when the detector detects the local magnetic field, or be provided with a display adapted to indicate when the detector detects the local magnetic field.

In accordance with a second embodiment of the invention, the magnetic detector is designed to be subcutaneously implanted in the patient at the implanted injection port, and the magnetic device is adapted to emit the local magnetic field through the patient's skin from outside the patient's body and is movable externally along the patient's body to establish the injection position where the local magnetic field is detected by the implanted magnetic detector. The movable magnetic device is suitably adapted to establish the injection position In front of the subcutaneously implanted injection post. In its simplest form, the implanted magnetic detector may be adapted to emit a sound when detecting the local magnetic field. In a more sophisticate form, a sender may be implantable in the patient's body and be capable of sending information about the magnetic to outside the patient's body, as the implanted magnetic detector detects the local magnetic field emitted by the movable magnetic device from outside the patient's body. For example, the implanted sender may send RF signals that inform when the implanted detector detects the local magnetic field, whereby an accurate injection position at the patient's skin can be established. The accurate injection position may be directly or indirectly correlated to the intensity of magnetism detected by the magnetic detector.

The magnetic device may be a solenoid or a permanent magnet, which is sending out a magnetic field.

The present invention also relates to a method of detecting a wireless injection port subcutaneously implanted in a patient. The method comprises providing a magnetic device capable of emitting a local magnetic field through the patient's skin, providing a magnetic detector adapted to detect the local magnetic field emitted by the magnetic device, subcutaneously implanting the magnetic device or magnetic detector in the patient at the implanted injection port, moving the magnetic detector or magnetic device externally along the patient's body, and establishing an injection position at the patient's skin where the local magnetic field emitted by the magnetic device is detected by the magnetic detector. Then, an injection needle can be placed in the injection position where the local magnetic field has been detected to accurately insert the needle through the patient's skin directly into the injection port.

In accordance with a first alternative of the method of the invention, the magnetic device is subcutaneously implanted, the magnetic detector is moved externally along the patient's body, and the injection position is established at the patient's skin where the local magnetic field emitted by the implanted magnetic device is detected by the moving magnetic detector.

In accordance with a second alternative of the method of the invention, the magnetic detector is subcutaneously implanted, the magnetic device is moved externally along the patient's body, and the injection position is established at the patient's skin where the local magnetic field emitted by the moving magnetic device is detected by the implanted magnetic detector. When practising the second alternative method it may further comprise Implanting a sender and using the sender to send information to outside the patient's body confirming when the implanted magnetic detector detects the local magnetic field emitted by the moving magnetic device.

When practising the above detection method a semiconductor circuit, preferably comprising at least one Hall-element, may be used as the magnetic detector.

The present invention also provides a surgical method for treating a patient having a disease comprising the steps of: insufflating the patient's abdomen with gas; implanting a hydraulically operable implant designed for treating reflux disease, urinary incontinence, impotence, anal incontinence or obesity in the abdomen by using surgical instruments through the trocars; subcutaneously implanting an injection port for supplying hydraulic fluid for the operation of the implant and a magnetic device at the injection port for emitting a local magnetic field through the injection port and the adjacent skin portion of the patient; postoperative moving an external magnetic detector along the patient's body to a position in which the local magnetic field emitted by the implanted magnetic device is detected by the magnetic detector; bringing an injection needle to the position in which the local magnetic field is detected; and moving the injection needle to penetrate the patient's skin into the injection port for supplying hydraulic fluid to or withdrawing hydraulic fluid from the injection port.

Alternatively, the surgical method may comprise subcutaneously implanting a magnetic detector at the injection port and post-operatively moving an exterior magnetic device emitting a local magnetic field along the patient's body to a position in which the local magnetic field emitted by the exterior magnetic device is detected by the implanted magnetic detector.

The invention is described in more detail in the following with reference to the accompanying drawings, in which

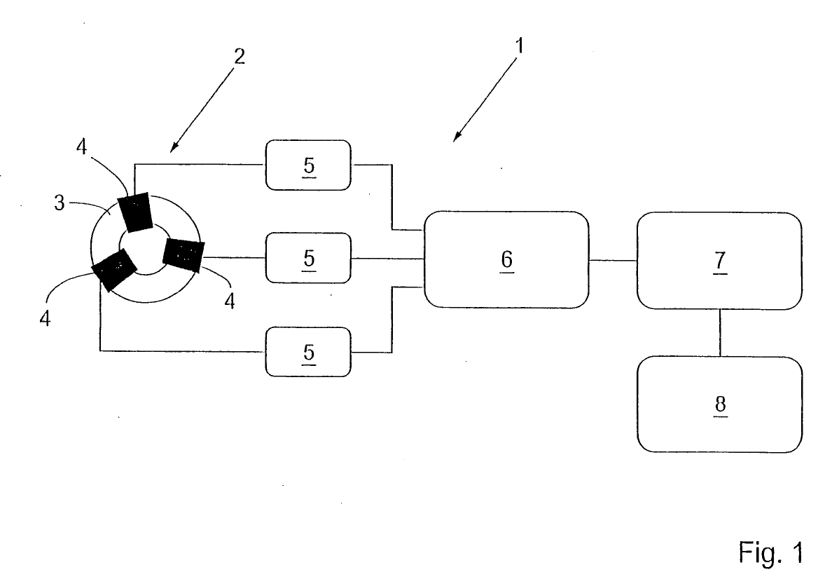

FIG. 1 shows a connection diagram for a magnetic detector of the apparatus according to the present invention,

FIG. 2 schematically illustrates in a diagram the output of the magnetic detector positioned in front of a magnetic device of the apparatus of the invention.

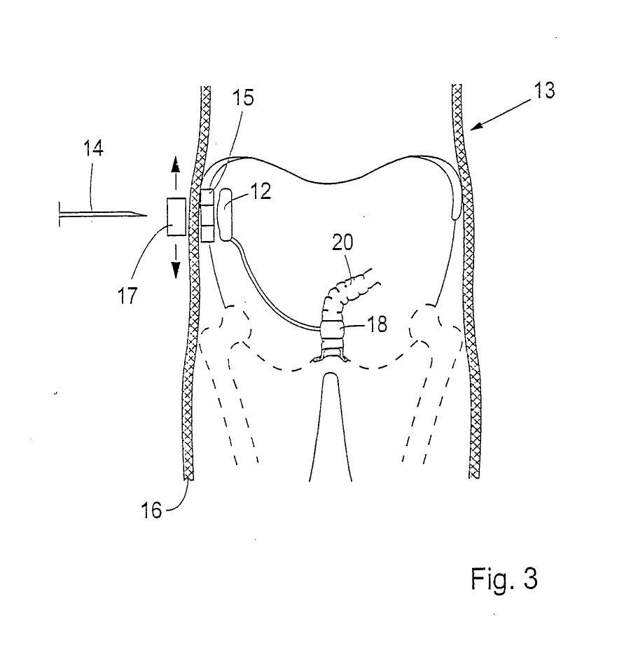

FIG. 3 is a schematic view of an embodiment where the magnetic device is subcutaneously implanted in a patient, and the magnetic detector is movable externally along the patient's body,

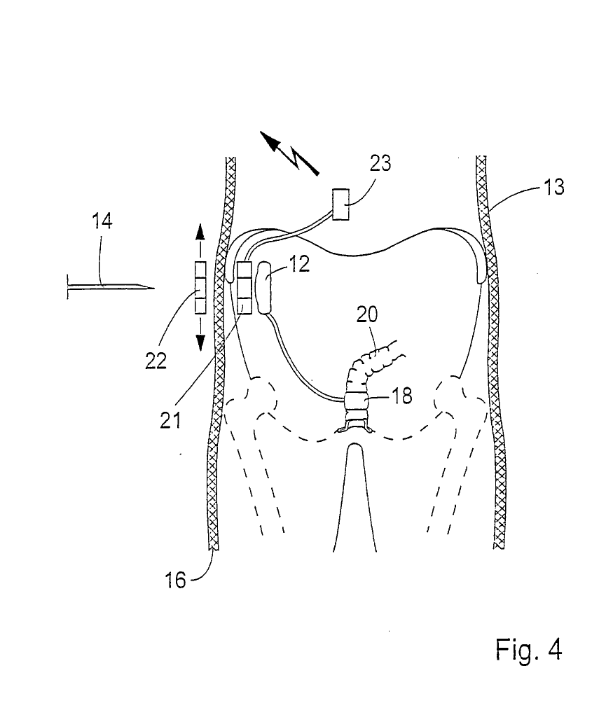

FIG. 4 is a schematic view of an embodiment where the magnet c detector is subcutaneously implanted in the patient and the magnetic device is movable externally along the patient's body,

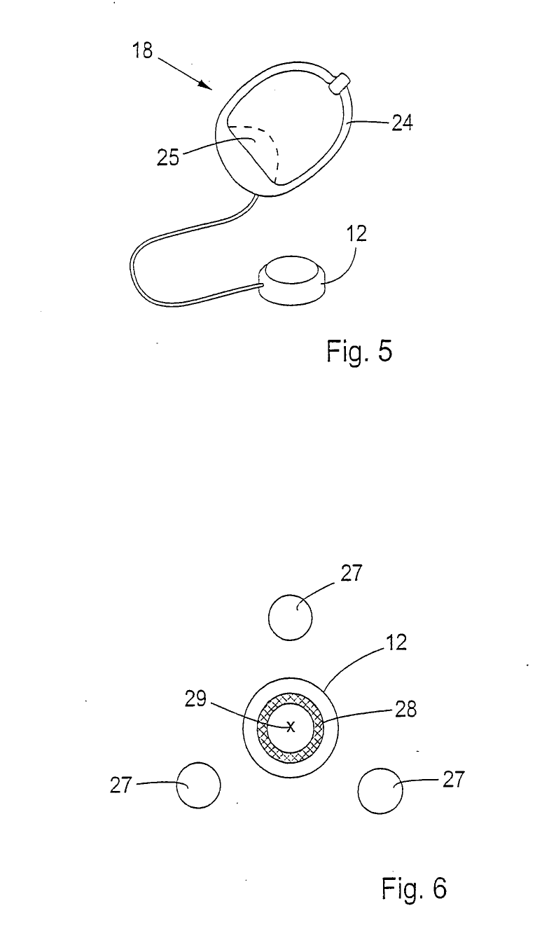

FIG. 5 is a schematic view of a hydraulically adjustable constriction device designed for treating reflux disease, urine incontinence, anal incontinence or obesity, and

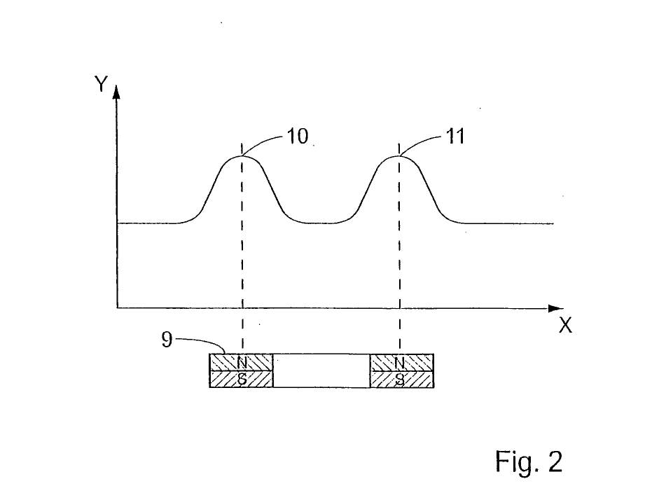

FIG. 6 illustrates an embodiment according to the present invention using Hall-elements as the magnetic detecting device.

Referring to the drawing figures, like reference numerals designate identical or corresponding elements throughout the several figures.

FIG. 1 shows a connection circuit 1 for a magnetic detector 2 of the apparatus according to the present invention. A magnetic device in the form of a ring-magnet 3, which can be a solenoid or a permanent magnet, is implanted in a patient's body. Located outside the body and positioned in front of the implanted ring-magnet 3 is magnetic detector 2, which includes three linear magnetic field sensors 4 grouped in a triangular configuration. Each sensor 4 includes a semiconductor circuit such as a Hall-element or the like. Sensors 4 are connected to signal-conditioning amplifiers 5, which in turn, are connected to an A/D-converter 6. A microprocessor 7 is connected to A/D-converter 6. To visually display the output signals of sensors 4, a display-device 8 is connected to microprocessor 7.

The graph shown in FIG. 2 illustrates, in principle, how the information obtained by detector 2 can be presented. On the X-axis in the graph is the position of detector 2 relative to the magnetic device. On the Y-axis is the combined output of sensors 4 of detector 2. Thus, the graph of FIG. 2 shows the position “X” of detector 2 relative to the magnetic device as a function of detector 2's output “Y”. To illustrate this method of detecting, a magnetic device in the form of a ring-magnet 9 is shown relative to the graph of FIG. 2. Ring-magnet 9 is shown in cross-section to show the positions of its magnetic north pole N and south pole S, respectively. FIG. 2 depicts the case where magnetic detector 2 (not shown in FIG. 2) has been centred in front of ring-magnet 9 and where all of the sensors 4 produce a maximum output, which is shown as peaks 10,11 in the graph of FIG. 2. Sensors 4 are connected (e.g., by connection circuit 1 shown in FIG. 1) to display device 8, which may display the graph shown in FIG. 2, or alternatively, a numeral result from the measurements taken by sensors 4.

FIG. 3 shows an embodiment of the apparatus of the present invention for detecting an infection port 12 subcutaneously implanted in a patient 13 suffering from anal incontinence to enable positioning of an injection needle 14 outside the patient's body for safe and accurate injection in the injection port 12. Injection port 12 is hydraulically connected to a hydraulically adjustable artificial sphincter 18 applied to the patient's rectum 20. The apparatus also includes a magnetic device in the form of a ring-magnet 15 subcutaneously implanted in the patient 13 around injection port 12. Magnetic device 15 emits a local magnetic field extending through a portion of the patient's 13 skin 16 adjacent to injection port 12. The apparatus further includes an external, separate magnetic detector 17 that may be manually moved along the patient 13's body to establish an injection position at the patient's skin where the local magnetic field emitted by magnetic device 15 is detected by magnetic detector 17. When this injection position has been established, injection needle 14 can be located in the same position to accurately insert the needle 14 through patient's skin directly into injection port 12.

FIG. 4 shows an embodiment of the invention identical to the embodiment according to FIG. 3, except that a magnetic detector 21 is subcutaneously implanted in patient 13 at injection port 12 and an external separate magnetic device in the form of a ring-magnet 22 that emits a local magnetic field through patient's 13 skin 16 is provided. Ring-magnet 22 may be manually moved externally along the patient's 13 body to establish an injection position at the patient's skin where the local magnetic field emitted by magnetic device 22 is detected by the implanted magnetic detector 21. A sender 23 is implanted in patient 13 and sends information about the status of magnetic detector 21. Thus, when magnetic detector 21 detects the local magnetic field emitted by external ring-magnet 22, sender 23 sends information confirming that magnetic device 22 is in the proper injection position for accurate positioning of the injection needle 14 outside the patient's body. When this injection position has been established, the injection needle 14 can be placed in the same position to accurately insert the needle through patient's skin directly into injection port 12.

FIG. 5 shows an example of the artificial sphincter 18 shown in FIGS. 3 and 4. Sphincter 18 includes a hydraulically adjustable constriction device 24 to be supplied around the patient's rectum (not shown in FIG. 5). Constriction device 24 has a cavity 25 which can be inflated by supplying hydraulic fluid thereto, via implanted injection port 12, to close the rectum, and be deflated by withdrawing hydraulic fluid therefrom, to open the rectum. This type of constriction device may also be used as an artificial sphincter for treating patient's suffering from heartburn and reflux disease or urinary incontinence, when combined with the apparatus of the present invention. Furthermore, constriction device 24 may be used for forming an adjustable stoma opening in the stomach or esophagus of an obese patient to treat obesity or for restricting the penile exit blood flow to treat an impotent patient, when combined with the apparatus of the invention.

FIG. 6 shows an advantageous design of one embodiment shown in FIG. 3, in which the external magnetic detector 17 includes three symmetrically arranged Hall-elements 27 which are grouped around a central point in a triangular configuration. The magnetic device is implanted and includes a ring-magnet 28 surrounding the centre 29 of the implanted injection port 12. When magnetic detector 17 is moved to position in which Hall-elements 27 are placed symmetrically above and around magnet 28, as illustrated in FIG. 6, magnetic detector 17 detects a maximum intensity of the magnetic field emitted by the implanted magnet 28, whereby the most accurate position where the injection needle 14 should be inserted into injection port 12 is established. As an alternative, the design described above may be practised in the embodiment shown in FIG. 4. Thus, the implanted magnetic detector 21 may include the three Hall-elements 27 and the external magnetic device 22 may include the ring-magnet 28.

Although the present invention has been described in terms of a particular embodiment and process, it is not intended that the invention be limited to that embodiment. Modifications of the embodiment and process within the spirit of the invention will be apparent to those skilled in the art. The scope of the invention is defined by the claims that follow.

1. An apparatus for detecting an injection port having a membrane and subcutaneously implanted in a patient, comprising:
a magnetic device for emitting a local magnetic field, and
a magnetic detector adapted for detecting the local magnetic field emitted by the magnetic device,
wherein the magnetic device or magnetic detector is designed for subcutaneously implantation in the patient at the implanted injection port, and the magnetic detector or magnetic device is movable externally along the patient's body to establish an injection position at the patient's skin in front of the implanted injection port where the local magnetic field emitted by the magnetic device is detected by the magnetic detector, whereby an injection needle can be placed in the established injection position, in order to insert the injection needle through the patient's skin directly into the injection port substantially in the centre thereof.

2. An apparatus according to claim 1, wherein the magnetic detector comprises a semiconductor circuit.

3. An apparatus according to claim 2, wherein the semiconductor circuit of the magnetic detector comprises at least one Hall-element.

4. An apparatus according to claim 3, wherein the magnetic detector comprises several Hall-elements grouped around a central point in a triangular or square-configuration.

5. An apparatus according to claims 1 wherein the magnetic device is designed for subcutaneously implantation in the patient at the implanted injection port to emit the local magnetic field through a portion of the patient's skin adjacent to the injection port, and the magnetic detector is movable externally along the patient's body to establish the injection position where the local magnetic field is detected by the magnetic detector.

6. An apparatus according to claim 5, wherein the magnetic device comprises a ring-magnet designed for implantation around the membrane of the implanted injection ports.

7. An apparatus according to claims 1, wherein the magnetic detector is designed for subcutaneously implantation in the patient at the implanted injection port, and the magnetic device is emits the local magnetic field through the patient's skin from outside the patient's body and is movable externally along the patient's body to establish the injection position where the local magnetic field is detected by the implanted magnetic detector.

8. An apparatus according to claim 7, wherein the magnetic device comprises a ring-magnet.

9. An apparatus according to claim 7, further comprising a sender implantable in the patient's body for sending information about the magnetic detector to outside the patient's body, as the magnetic detector detects the local magnetic field emitted by the magnetic device from outside the patient's body.

10. An apparatus according to claim 1, wherein the magnetic detector emits a sound when detecting the local magnetic field.

11. An apparatus according to claim 2, wherein the magnetic detector is provided with at least one diode for emitting light when the detector detects the local magnetic field.

12. An apparatus according to claim 2, wherein the magnetic detector is provided with a display for indicating when the detector detects the local magnetic field.

13. An apparatus according to claim 1, wherein the magnetic device is a solenoid or a permanent magnet.

14. Use of the apparatus according to claim 1 for detecting a subcutaneously implanted injection port, which is hydraulically connected to an implanted hydraulically adjustable constriction device for treating reflux disease, obesity, anal or urinary incontinence, or impotence.

15. A method of detecting an injection port subcutaneously implanted in a patient, comprising:
providing a magnetic device for emitting a local magnetic field through the patient's skins,
providing a magnetic detector for detecting the local magnetic field emitted by the magnetic device,
subcutaneously implanting the magnetic device or magnetic detector in the patient at the implanted injection port,
moving the magnetic detector or magnetic device externally along the patient's body, and
establishing an injection position at the patient's skin in front of the implanted injection port where the local magnetic field emitted by the magnetic device is detected by the magnetic detector.

16. A method according to claim 15, wherein the magnetic device is subcutaneously implanted, the magnetic detector is moved externally along the patient's body, and the injection position is established at the patient's skin where the local magnetic field emitted by the implanted magnetic device is detected by the moving magnetic detector.

17. A method according to claim 15, wherein the magnetic detector is subcutaneously implanted, the magnetic device is moved externally along the patient's body, and the injection position is established at the patient's skin where the local magnetic field emitted by the moving magnetic device is detected by the implanted magnetic detector.

18. A method according to claim 17, further comprising implanting a sender and using the sender to send information to outside the patient's body confirming when the implanted magnetic detector detects the local magnetic field emitted by the exterior magnetic device.

19. A method according to claim 15, wherein a semiconductor circuit is used as the magnetic detector.

20. A method according to claim 19, wherein the semiconductor circuit comprises at least one Hall-element.

21. A surgical method for treating a patient having a disease comprising the steps of:
insufflating the patient's abdomen with gas;
implanting a hydraulically operable implant designed for treating reflux disease, urinary incontinence, impotence, anal incontinence or obesity in the abdomen by using surgical instruments through the trocars;
subcutaneously implanting an injection port for supplying hydraulic fluid for the operation of the implant and a magnetic device at the injection port for emitting a local magnetic field through the injection port and the adjacent skin portion of the patient;
post-operatively moving an exterior magnetic detector along the patient's body to a position in which the local magnetic field emitted by the implanted magnetic device is detected by the magnetic detector;
bringing an injection needle to the position in which the local magnetic field is detected; and
moving the injection needle to penetrate the patient's skin into the injection port for supplying hydraulic fluid to or withdrawing hydraulic fluid from the injection port.

22. A surgical method for treating a patient having a disease comprising the steps of:
insufflating the patient's abdomen with gas;
implanting a hydraulically operable implant designed for treating reflux disease, urinary incontinence, impotence, anal incontinence or obesity in the abdomen by using surgical instruments through the trocars;
subcutaneously implanting an injection port for supplying hydraulic fluid for the operation of the implant and a magnetic detector at the injection port;
post-operatively moving an exterior magnetic device emitting a local magnetic field along the patient's body to a position in which the local magnetic field emitted by the exterior magnetic device is detected by the implanted magnetic detector;
bringing an injection needle to the position in which the local magnetic field is detected; and
moving the injection needle to penetrate the patient's skin into the injection port for supplying hydraulic fluid to or withdrawing hydraulic fluid from the injection port.
